(19) United States
(12) Patent Application Publication
(10) Pub. No.: US 2023/0372312 A1
(43) Pub. Date: Nov. 23, 2023

(54) Methods of Treating Cushing's syndrome and Liver Disorders, and of Reducing Liver Toxicity of Other Drugs Administered to a Patient

(71) Applicant: Corcept Therapeutics Incorporated, Menlo Park, CA (US)

(72) Inventor: Andreas Moraitis, Sunny Isles Beach, FL (US)

(73) Assignee: Corcept Therapeutics Incorporated, Menlo Park, CA (US)

(21) Appl. No.: 18/199,624

(22) Filed: May 19, 2023

Related U.S. Application Data
(60) Provisional application No. 63/344,308, filed on May 20, 2022.

(51) Int. Cl. A61K 31/437; A61K 45/06; A61P 5/38; A61K 31/4196

(52) U.S. Cl. CPC A61K 31/437; A61K 45/06; A61P 5/38; A61K 31/4196

(57) ABSTRACT

Methods and uses are disclosed for treating a subject suffering from a disorder selected from a liver disorder, Cushing's syndrome, or Cushing's Disease, cancer, an infection, an inflammatory condition, a cardiovascular, endocrine, or kidney disease, and combinations thereof, or other disorder for which they may be administered a drug which may cause liver toxicity, without adverse effects on the liver. Such liver disorders include fatty liver diseases are effective for reducing high levels of liver enzymes with a favorable safety profile. The methods and uses comprise administering to the subject an effective amount of a selective nonsteroidal glucocorticoid receptor modulator such as relacorilant, including methods and uses in combination with another drug, without adverse effects on liver enzyme levels, or on liver function. In embodiments, the other drug may be a drug that may cause liver toxicity, such as drugs that inhibit CYP3A enzymes, e.g., itraconazole or ketoconazole.

### CROSS-REFERENCE TO RELATED APPLICATION

This application claims priority to, and the benefit of, U.S. Provisional Application No. 63/344,308 filed May 20, 2022, which application is hereby incorporated by reference herein its entirety.

### BACKGROUND

Relacorilant (also known as CORT125134) binds the glucocorticoid receptor type 2 (GR), and is a highly selective GR modulator (SGRM). It is in clinical development for the treatment of: endogenous hypercortisolism (Cushing's syndrome (CS), including Cushing's Disease (CD)) of all etiologies (monotherapy), adrenocortical carcinoma and other solid tumors (with anticancer agents). Relacorilant lacks affinity for the progesterone receptor (unlike the FDA-approved GR antagonist mifepristone). Studies of Cushing's syndrome patients show that relacorilant administration can provide clinically meaningful improvement in hypertension and hyperglycemia, without antiprogesterone effects or drug-induced hypokalemia. Improvement in other cortisol-excess-related comorbidities observed, including hypercoagulopathy, cognitive function, mood, and quality of life in these patients have also been observed.

Elimination of administered relacorilant is primarily hepatic (via CYP3A and carbonyl reductase). It is a strong CYP3A4 inhibitor, but its administration does not result in clinically significant inhibition of CYP2C8 or CYP2C9. Thus, it appears to interact with liver and other enzymes.

However, many drugs which affect the glucocorticoid receptor (GR), or cortisol levels or cortisol effects have significant impacts on the liver. Many patients are administered drugs which may cause liver toxicity. For example, some drugs, such as ketoconazole and itraconazole may lead to liver toxicity. However, there are patients who may be administered both a drug which may cause liver toxicity and also a drug which affects the GR. For example, excess cortisol levels, as are often found with Cushing's syndrome, can lead to, or are often accompanied by, high levels of liver fat, high levels of liver enzymes (such as, e.g., alanine amino transferase (ALT), asparate amino transferase (AST) and others), liver steatohepatitis (NASH), and other liver disorders. Such excess cortisol levels may be treated with drugs which affect the GR.

Liver disorders can be categorized in different groups of diseases, such as alcohol-induced fatty liver disease (AFLD), nonalcoholic fatty liver disease (NAFLD), drug- or alcohol-related liver diseases, viral diseases, immune-mediated liver diseases, metabolic liver diseases, and complications associated with hepatic insufficiency and/or liver transplantation. Nonalcoholic fatty liver disease is a common hepatic disorder with histological features similar to those of alcohol-induced fatty liver disease, in individuals who consume little or no alcohol. Fatty liver disease is due to an abnormal retention of lipid (fats) within hepatocytes. Effective treatments for AFLD and NAFLD remain insufficient. To date, no therapeutic drug treatment is established for such patients. There is a need for novel therapeutic options for managing fatty liver disease. There is a need for novel therapeutic options for patients in need of treatment by both a drug which may cause liver toxicity and a drug which affects the GR.

### SUMMARY

The present methods provide improved methods of administering selective glucocorticoid receptor modulators (SGRMs) and drugs that may cause liver toxicity to patients in need thereof. Disclosed herein are novel methods for treating Cushing's syndrome and Cushing's Disease with a favorable liver safety profile. Also disclosed herein are novel methods for treating liver disorders including fatty liver diseases, for reducing high levels of liver enzymes, and other treatments, all with a favorable liver safety profile. Applicant discloses herein the favorable liver safety profile of the SGRM relacorilant following administration to healthy and to hepatically impaired adults, and to patients with Cushing's syndrome. The methods comprise administering to the subject an effective amount of relacorilant without adverse effects on liver enzyme levels, or on liver function. In embodiments, the methods comprise administering to the subject an effective amount of relacorilant in combination with another drug, without adverse effects on liver enzyme levels, or on liver function. In embodiments, the methods include methods of reducing or preventing liver toxicity in a patient receiving a drug which may cause liver toxicity, the method comprising administering to the patient an effective amount of a nonsteroidal selective glucocorticoid receptor modulator (SGRM) in addition to said drug which may cause liver toxicity. In embodiments, the patient is receiving a drug which may cause liver toxicity, and is then administered a SGRM. In embodiments, the patient is receiving a SGRM, and is then administered a drug which may cause liver toxicity. In embodiments, the other drug may be a drug that may inhibit CYP3A enzymes (including CYP3A4 enzymes), such as, e.g., itraconazole or ketoconazole. In embodiments, the methods comprise reducing liver steatosis in a patient suffering from a liver disorder, Cushing's syndrome, Cushing's Disease, or a combination thereof, by administering to the patient an effective amount of a nonsteroidal selective glucocorticoid receptor modulator (SGRM) effective to reduce said liver steatosis in said patient. In embodiments, the methods comprise co-administering an effective amount of relacorilant when the patient is being, or is to be, administered a drug with known liver toxicity (e.g., itraconazole, ketoconazole, or other CYP3A inhibitor) effective to reduce, prevent, or abolish the liver toxicity of that drug with known liver toxicity.

Relacorilant is a heteroaryl-ketone fused azadecalin compound disclosed and described in Example 18 of U.S. Pat. No. 8,859,774, the entire contents of which U.S. patent is hereby incorporated by reference in its entirety. The chemical name of relacorilant is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone; it has the following structure:

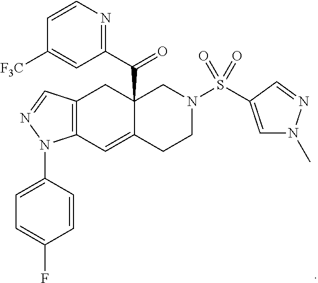 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Applicant has discovered and discloses herein that relacorilant has a favorable liver safety profile that includes a trend toward improved liver function tests (LFTs) in healthy volunteers and patients with normal and impaired liver function. Applicant has discovered and discloses herein that relacorilant may be safely administered to patients with moderate hepatic impairment as well as to patients with normal liver function. Thus, SGRMs such as relacorilant may be administered to patients with impaired liver function in the same manner, dosing, and frequency of administration as it is administered to patients with normal liver function. The findings in patients with liver impairment support the use of relacorilant in treating Cushing's syndrome and Cushing's Disease patients who have moderate hepatic impairment without need for adjustment of the relacorilant dose (as compared to the dose used for patients without moderate hepatic impairment). The findings in patients with liver impairment also support the use of relacorilant in treating patients suffering from liver disorders who have moderate hepatic impairment without need for adjustment of the relacorilant dose (as compared to the dose used for patients without liver disorders). Relacorilant may be administered in the treatment of liver disorders including, for example, a fatty liver disorder. Fatty liver disorders include, for example, alcohol-related liver disease, and nonalcoholic fatty liver disease (NAFLD), non-alcoholic steatohepatitis (NASH), and other liver disorders. A SGRM such as relacorilant may be administered to a patient suffering from cancer, or from a fungal infection, or from another disorder for which a drug that may cause liver toxicity may be co-administered, without need for adjustment of the relacorilant dose (as compared to the dose used in the absence of the drug that may cause liver toxicity).

Relacorilant may be orally administered, or may be administered by other suitable means. In the studies disclosed herein, relacorilant was orally administered in doses of 100 milligrams (mg) to 400 mg per day. Relacorilant may be administered once daily, or may be administered twice, or three times, or other number of times per day.

In embodiments of the methods disclosed herein, the effective amounts of relacorilant may include a daily dose of between 1 and 100 mg/kg/day. In some embodiments, the daily relacorilant dose is 1, 2, 4, 6, 8, 10, 12, 14, 16, 18, 20, 30, 40, 50 60, 70, 80, 90 or 100 mg/kg/day. Relacorilant may be administrated for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 weeks.

The present methods provide improved methods of treating patients who are administered an SGRM and a drug that may cause liver toxicity to patients. The present methods provide improved methods of treating Cushing's syndrome, Cushing's Disease, and liver disorders including fatty liver disease, excess liver enzyme levels, and other liver disorders. The present methods provide improved methods for reducing liver steatosis in a patient suffering from a liver disorder, Cushing's syndrome, Cushing's Disease, or a combination thereof. The present methods provide improved methods for reducing, preventing, or abolishing the liver toxicity of drugs with known liver toxicity by administration of a SGRM, such as relacorilant, with the drug with known liver toxicity.

### BRIEF DESCRIPTION OF THE DRAWINGS

FIG. 1 shows the results of the hepatic impartment study.

FIG. 2 shows the results of the drug-drug interaction study (relacorilant-itraconazole).

FIG. 3 shows the results of the Phase 2 Cushing's syndrome study.

FIG. 4 shows the study designs of on-going clinical studies.

### DETAILED DESCRIPTION

Methods and uses are disclosed for treating a subject suffering from a disorder selected from a liver disorder, Cushing's syndrome, or Cushing's Disease, cancer, an infection, an inflammatory condition, a cardiovascular, endocrine, or kidney disease, and combinations thereof, or other disorder for which they may be administered a drug which may cause liver toxicity, without adverse effects on the liver. Such liver disorders include fatty liver diseases are effective for reducing high levels of liver enzymes with a favorable safety profile. The methods and uses comprise administering to the subject an effective amount of a selective nonsteroidal glucocorticoid receptor modulator such as relacorilant, including methods and uses in combination with another drug, without adverse effects on liver enzyme levels, or on liver function. In embodiments, the other drug may be a drug that may cause liver toxicity, such as drugs that inhibit CYP3A enzymes, e.g., itraconazole or ketoconazole.

Accordingly, Applicant discloses herein improved methods for administering a nonsteroidal selective glucocorticoid receptor modulator (SGRM) to a patient who may be administered another drug which may cause liver toxicity, without adverse effects on the liver of the patient. The SGRM may be, e.g., a heteroaryl-ketone fused azadecalin compound, and in embodiments may be relacorilant. Applicant discloses herein that a SGRM, such as relacorilant, may be administered at the same dose to either a patient with normal liver or to a patient with moderate liver impairment. Applicant discloses herein that a SGRM, such as relacorilant, may be administered at the same dose to either a patient who is not receiving a drug which may cause liver toxicity, or to a patient who is, or will, receive a drug which may cause liver toxicity. SGRMs such as relacorilant may be administered to patients with impaired liver function in the same manner, dosing, and frequency of administration as it is administered to patients with normal liver function.

The methods disclosed herein include methods of treating a patient receiving, or soon to receive, a drug which may cause liver toxicity, without adverse effects on the liver of said patient, the method comprising administering to the patient an effective amount of a nonsteroidal selective glucocorticoid receptor modulator (SGRM) to treat said patient receiving, or soon to receive, said drug which may cause liver toxicity, without adverse effects on the liver of the patient. In embodiments, the methods include methods wherein said patient is receiving said drug which may cause liver toxicity, and is then administered said SGRM. In embodiments, the methods include methods wherein said patient is receiving said SGRM, and is then administered said drug which may cause liver toxicity. In embodiments, the SGRM is relacorilant. In embodiments, the patient suffers from Cushing's syndrome, Cushing's Disease, a liver disease, a cancer, or other disorder for which the patient may be administered a drug which may cause liver toxicity.

Applicants disclose herein uses of a SGRM, such as relacorilant, for reducing or preventing liver toxicity in a patient receiving, or soon to receive, a drug which may cause liver toxicity, the uses comprising the use of an effective amount of a nonsteroidal selective glucocorticoid receptor modulator (SGRM) in combination (before, during, or after administration of) with a drug which may cause liver toxicity. In embodiments, the patient has received a drug which may cause liver toxicity, and the SGRM is then used. In embodiments, the patient has received a SGRM, and the drug which may cause liver toxicity is then used. In embodiments, the SGRM is relacorilant.

The methods and uses disclosed herein can be used to treat a patient suffering from Cushing's syndrome, Cushing's Disease, or a liver disorder by administering an effective amount of a glucocorticoid receptor modulator (GRM), preferably a selective glucocorticoid receptor modulator (SGRM). In embodiments, GRM administration may be in combination with other pharmaceuticals or medical treatment effective to treat the liver disorder. In preferred embodiments, the SGRM is a nonsteroidal SGRM, such as a compound comprising a fused azadecalin structure. In embodiments, the nonsteroidal SGRM is a compound comprising a heteroaryl ketone fused azadecalin structure.

Applicant discloses herein methods of treating a patient suffering from Cushing's syndrome, Cushing's Disease, a liver disorder, or cancer, or combinations thereof, without adverse effects on the liver of said patient, the method comprising administering to the patient an effective amount of a nonsteroidal selective glucocorticoid receptor modulator (SGRM) to treat said liver disorder or Cushing's syndrome without adverse effects on the liver of the patient. Applicant discloses herein methods of treating a patient suffering from, and receiving medication for, Cushing's syndrome, Cushing's Disease, a liver disorder, cancer, a fungal infection, a bacterial infection, a viral infection, an inflammatory disease or condition, a cardiovascular disease, an endocrine condition, a kidney disease, or combinations thereof, without adverse effects on the liver of said patient, the method comprising administering to the patient an effective amount of a nonsteroidal selective glucocorticoid receptor modulator (SGRM) to treat said disorder without adverse effects on the liver of the patient.

Applicant discloses herein methods of reducing liver steatosis in a patient suffering from a liver disorder, Cushing's syndrome, Cushing's Disease, or a combination thereof, the method comprising administering to the patient an effective amount of a nonsteroidal selective glucocorticoid receptor modulator (SGRM) to reduce said liver steatosis in said patient.

Applicant discloses herein methods of method of reducing or preventing liver toxicity in a patient receiving a drug which may cause liver toxicity, the method comprising administering to the patient an effective amount of a nonsteroidal selective glucocorticoid receptor modulator (SGRM) in addition to said drug which may cause liver toxicity. Many different drugs have been implicated in liver toxicity (see, e.g., Bjornsson et al., Int. J. Mol. Sci. 2016, 17, 224-230; and Chen et al., Drug Discov Today. 2016, 21(4): 648-653). A ranked list of drugs (ranked by order of severity of liver injury) is available for download from the U.S. Food and Drug Administration website “fda.gov” entitled “Drug Induced Liver Injury Rank (DILIrank) Dataset”). The DILIrank dataset consists of 1,036 FDA-approved drugs that are divided into three classes according to their potential for causing drug-induced liver injury (DILI), and a fourth group of drugs for which the data regarding the liver toxicity is ambiguous. Of the 1036 drugs discussed, 192 were deemed to be of the most concern, and 278 were deemed to be of lesser concern as compared to the most concerning drugs; for 254 drugs the analysis was ambiguous, and 312 of the 1036 were found to be of no concern regarding Drug-induced liver injury.

In embodiments, the drug which may cause liver toxicity is a drug listed in the DILIrank dataset. In embodiments, the drug which may cause liver toxicity is a drug identified as being of “Most-DILI-Concern” or of “Less-DILI-Concern” in the DILIrank dataset. In embodiments, the drug which may cause liver toxicity is a drug identified as being of “Most-DILI-Concern” in the DILIrank dataset. The 192 drugs identified as being of Most-DILI Concern in the DILIrank Dataset are: mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, exemestane, gefitinib, eltrombopag olamine, oxaliplatin, diltiazem, estramustine, peginterferon alfa-2b, methotrexate, cytarabine, maraviroc, mexiletine, and pentostatin.

In embodiments, the drug which may cause liver toxicity is a CYP3A inhibitor. In embodiments, the drug which may cause liver toxicity is ketoconazole, or is itraconazole. In embodiments, the drug which may cause liver toxicity is selected from ketoconazole, itraconazole, nefazodone, ritonavir, nelfinavir, indinavir, boceprevir, clarithromycin, conivaptan, lopinavir, posaconazole, saquinavir, telaprevir, cobicistat, troleandomycin, tipranivir, paritaprevir and voriconazole.

In embodiments, the patient suffers from Cushing's syndrome. In embodiments, the patient suffers from Cushing's syndrome and a liver disorder.

In embodiments, the liver disorder is a fatty liver disease. In embodiments, the fatty liver disease is selected from alcohol related liver disease (ARLD) and nonalcoholic fatty liver disease (NAFLD). In embodiments, the alcohol-related liver disease (ARLD) is alcohol fatty liver disease (AFL), alcoholic steatohepatitis (ASH) or alcoholic cirrhosis. In embodiments, the nonalcoholic fatty liver disease (NAFLD) is nonalcoholic steatohepatitis (NASH) or nonalcoholic cirrhosis.

The nonsteroidal selective glucocorticoid receptor modulator may be a compound comprising a heteroaryl ketone fused azadecalin structure. In embodiments, the nonsteroidal selective glucocorticoid receptor modulator is relacorilant, (R)-(1-(4-fluorophenyl)-6-(1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, having the formula:

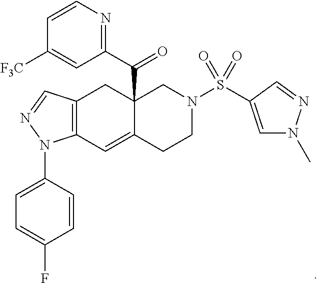 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

The methods disclosed herein may further comprise administering a further pharmaceutical composition to the patient, without further adverse effects on the liver of said patient. In embodiments the further pharmaceutical composition comprises itraconazole or ketoconazole. In embodiments the further pharmaceutical composition comprises a CYP3A inhibitor. In embodiments the further pharmaceutical composition comprises a CYP3A inhibitor selected from ketoconazole, itraconazole, nefazodone, ritonavir, nelfinavir, indinavir, boceprevir, clarithromycin, conivaptan, lopinavir, posaconazole, saquinavir, telaprevir, cobicistat, troleandomycin, tipranivir, paritaprevir and voriconazole. In embodiments the further pharmaceutical composition comprises a drug selected from mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, alfa-2b, methotrexate, cytarabine, maraviroc, mexiletine, and pentostatin.

Current clinical studies of relacorilant in patients with endogenous hypercortisolism (e.g., Cushing's syndrome of Cushing's Disease patients) include liver function test (LFT) assessments. In embodiments, the LFT assessments include measurements of alanine aminotransferase (ALT) levels, measurements of aspartate aminotransferase (AST) levels, or both. LFT assessments may further include magnetic resonance images (MM) of the liver, computer aided tomography (CAT) images of the liver, liver biopsies, and other tests. Other clinical parameters of the patient may be determined, including, e.g., blood pressure, blood glucose levels, Hb1Ac levels, blood potassium levels, body weight, glucose tolerance measurements, insulin levels, blood coagulation measurements, patient cognitive function, patient mood, quality of life, and other measurements.

### B. Definitions

Cushing's syndrome is a condition caused by the excessive production of the glucocorticoid cortisol by the adrenal cortex. The condition is often due to the presence of a tumor or hyperplasia that exhibits unregulated secretion of adrenocorticotropic hormone (ACTH). The unregulated secretion of ACTH in turn induces the adrenal glands to secrete excess cortisol. Cortisol generally participates in a negative feedback loop, in which high levels of cortisol suppress secretion of both ACTH and cortisol. However, in Cushing's syndrome, this negative regulation is not effective or absent, resulting in chronic hypercortisolemia.

Liver enzymes are crucial to the utilization of food, maintenance of homeostasis of an organism (including a human patient), and may play an important role in metabolism of pharmaceutical compounds administered to a patient. Such liver enzymes, which may be present in other tissues as well, CYP enzymes such as CYP2C8, CYP2C9, CYP3A4, and other CYP enzymes, and include alanine aminotransferase (ALT), aspartate aminotransferase (AST). Such enzymes are discussed, for example, in U.S. Pat. Nos. 10,195,214 and 11,285,145, hereby incorporated by reference herein in their entireties.

As used herein, the terms “liver toxicity” and “hepatotoxicity” refer to liver damage and/or toxic liver disease caused by administration of drugs (including medications and herbal remedies), or exposure to solvents, or ingestion of toxic foods (e.g., toxic mushrooms), or other bodily insults that may damage the liver.

As used herein, the terms “drug which may cause liver toxicity”, “agent which may cause liver toxicity”, “hepatotoxic drug”, and “hepatotoxic agent” refer to drugs and agent that may cause liver damage and/or toxic liver disease. Such drugs and agents may be, without limitation, for example, medications, herbal remedies, solvents, toxic foods (e.g., toxic mushrooms), and other agents that may damage the liver.

Drugs which may cause liver toxicity include, without limitation, mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, alfa-2b, methotrexate, cytarabine, maraviroc, mexiletine, and pentostatin.

As used herein, the terms “liver disorder” and “liver disease” refer to a disorder or disease of the liver. Liver disorders include, without limitation, fatty liver disease, alcohol-related liver disease, non-alcoholic fatty liver disease, hepatitis, and other liver disorders. Liver disorders are discussed, for example, in U.S. Pat. No. 10,238,659, hereby incorporated by reference herein in its entirety.

“Fatty liver disease” refers to a disease or a pathological condition caused by, at least in part, abnormal hepatic lipid deposits. Fatty liver disease includes, e.g., alcoholic fatty liver disease, nonalcoholic fatty liver disease, and acute fatty liver of pregnancy. Fatty liver disease may be, e.g., macrovesicular steatosis or microvesicular steatosis.

“Alcohol-related liver disease” or “ARLD” refers to diseases of the liver that are wholly, or in part, caused by, or attributable to, excessive consumption of alcohol. There are four main types of ARLD, alcoholic fatty liver (AFL, a sub-type of fatty liver disease), alcoholic steatohepatitis (ASH), alcoholic-induced cirrhosis, and alcoholic hepatocellular cancer. As used herein, “excessive consumption of alcohol” generally refers to the consumption of more than about 15-30 g/day of ethanol.

The physiological effects of alcohol consumption on liver function or disease are dependent on a variety of genetic and non-genetic factors that modify both individual susceptibility and the clinical course of ARLD. Thus, in certain patients, ARLD can develop at much lower rates of alcohol consumption, including consumption of at least about 12 g/day, 15 g/day, 20 g/day, 25 g/day or more. Moreover, it is understood that in some patients, estimates of daily consumption of alcohol are an average value that includes periods of heavy alcohol consumption and periods of little or no alcohol consumption. Such an average value can include an average of alcohol consumption over at least about a week, two weeks, a month, three months, six months, nine months, a year, 2, 3, or 4 years, or more. In some cases, the determination of whether a liver dysfunction is an ARLD is based on reference to a variety of factors including, but not limited to: the amount and type of alcoholic beverage consumption (e.g., beer or spirits); the duration of alcohol abuse; patterns of drinking behavior (e.g., binge drinking, drinking without co-consumption of food, etc.); gender; ethnicity; co-existing disease conditions such as metabolic syndrome or diabetes, iron overload, or infection with hepatitis virus, genetic markers; family history; liver enzyme levels; proinflammatory cytokine levels; gene or protein expression analysis; or histopathological examination of liver tissue or cells.

“Liver disorder unrelated to excessive ingestion of alcohol” is a liver disorder that is distinguished from ARLD. Such a disorder therefore refers to a wide array of liver diseases that are not caused by alcohol consumption. For example, hepatitis can be caused by viral infection. A liver disorder caused by excessive alcohol consumption and other factors, is considered an ARLD rather than a liver disorder unrelated to excessive ingestion of alcohol. In contrast, a liver disorder merely exacerbated by excessive alcohol consumption is considered a liver disorder unrelated to excessive ingestion of alcohol.

“Nonalcoholic fatty liver disease” or “NAFLD” refers to a fatty liver disease characterized by the presence of fat (lipids) in the liver and no substantial inflammation or liver toxicity. NAFLD can progress into nonalcoholic steatohepatitis and then into irreversible, advanced liver scarring or cirrhosis.

“Nonalcoholic steatohepatitis” or “NASH” refers a fatty liver disease, which resembles alcoholic liver disease, but occurs in people who drink little or no alcohol. The major feature in NASH is fat in the liver, along with inflammation and damage. NASH can lead to cirrhosis, in which the liver is permanently damaged and scarred and is no longer able to function properly. A differential diagnosis of NASH versus NAFLD may be determined by liver biopsy.

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. Administration may be by oral administration (i.e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramusucular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art. The effective amount can be an amount effective to invoke an antitumor response. For the purpose of this disclosure, the effective amount of SGRM or the effective amount of a chemotherapeutic agent is an amount that would treat a liver disorder or bring about other desired beneficial clinical outcomes related to treatment of a liver disorder.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. In some cases, both agents are administered intermittently. In some embodiments, the one pharmaceutical agent, e.g., a SGRM, is administered daily, and the other pharmaceutical agent, e.g., a chemotherapeutic agent, is administered every two, three, or four days.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “composition” is intended to encompass a product comprising the specified ingredients such as a compound disclosed herein, and tautomeric forms, derivatives, analogues, stereoisomers, polymorphs, deuterated species, pharmaceutically acceptable salts, esters, ethers, metabolites, mixtures of isomers, pharmaceutically acceptable solvates thereof, and pharmaceutically acceptable compositions in specified amounts, as well as any product which results, directly or indirectly, from combination of the specified ingredients in the specified amounts. The pharmaceutical compositions discussed herein are meant to encompass any composition made by admixing compounds discussed and their pharmaceutically acceptable carriers.

As used herein, the terms “pharmaceutically-acceptable excipient” and “pharmaceutically acceptable carrier” are intended to include any and all solvents, dispersion media, coatings, antibacterial and antifungal agents, isotonic and absorption delaying agents, and the like, compatible with pharmaceutical administration. These terms refer to any substance that aids the administration of an active agent to—and absorption by—a subject and can be included in pharmaceutical compositions without causing a significant adverse toxicological effect on the patient. Except insofar as any conventional media or agent is incompatible with the active compound, use thereof in pharmaceutical compositions is contemplated. Non-limiting examples of pharmaceutically-acceptable excipients include water, NaCl, normal saline solutions, lactated Ringer's, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients may be useful as well.

As used herein, the phrase “nonsteroidal backbone” in the context of SGRMs refers to SGRMs that do not share structural homology to, or are not modifications of, cortisol with its steroid backbone containing seventeen carbon atoms, bonded in four fused rings. Such compounds include synthetic mimetics and analogs of proteins, including partially peptidic, pseudopeptidic and non-peptidic molecular entities.

The term “cortisol” refers to the naturally occurring glucocorticoid hormone (also known as hydrocortisone) that is produced by the zona fasciculata of the adrenal gland.

The terms “glucocorticosteroid” or “glucocorticoid” (“GC”) refer to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to any composition or compound which modulates GC binding to GR, or modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor modulator binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor modulator binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRM.

“Glucocorticoid receptor antagonist” (GRA) refers to any compound which inhibits GC binding to GR, or which inhibits any biological response associated with the binding of GR to an agonist. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. A GRA is a compound with an IC₅₀ (half maximal inhibition concentration) of less than 10 micromolar. See Example 1 of U.S. Pat. No. 8,859,774, the entire contents of which is hereby incorporated by reference in its entirety.

As used herein, the term “selective glucocorticoid receptor antagonist” (SGRA) refers to any composition or compound which inhibits GC binding to GR, or which inhibits any biological response associated with the binding of a GR to an agonist (where inhibition is determined with respect to the response in the absence of the compound). By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor antagonist bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor antagonist binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor antagonist binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRA.

Nonsteroidal GRA, SGRA, GRM, and SGRM compounds include compounds comprising a fused azadecalin structure (which may also be termed a fused azadecalin backbone), compounds comprising a heteroaryl-ketone fused azadecalin structure (which may also be termed a heteroaryl-ketone fused azadecalin backbone), compounds comprising an octahydro fused azadecalin structure (which may also be termed an octahydro fused azadecalin backbone), and compounds comprising a pyrimidine cyclohexyl backbone.

Nonsteroidal GRA, SGRA, GRM, and SGRM compounds include compounds comprising a fused azadecalin structure (which may also be termed a fused azadecalin backbone), compounds comprising a heteroaryl ketone fused azadecalin structure (which may also be termed a heteroaryl ketone fused azadecalin backbone), compounds comprising an octahydro fused azadecalin structure (which may also be termed an octahydro fused azadecalin backbone), and compounds comprising a pyrimidine cyclohexyl backbone. Exemplary nonsteroidal GRA, SGRA, GRM, and SGRM compounds comprising a fused azadecalin structure include those described in U.S. Pat. Nos. 7,928,237 and 8,461,172. Exemplary nonsteroidal GRA, SGRA, GRM, and SGRM compounds comprising a heteroaryl ketone fused azadecalin structure include those described in U.S. Pat. No. 8,859,774. Exemplary nonsteroidal GRA, SGRA, GRM, and SGRM compounds comprising an octahydro fused azadecalin structure include those described in U.S. Pat. No. 10,047,082. Exemplary nonsteroidal GRA, SGRA, GRM, and SGRM compounds comprising a pyrimidine cyclohexyl backbone include compounds disclosed in U.S. Pat. No. 8,685,973. All patents, patent publications, and patent applications disclosed herein are hereby incorporated by reference in their entireties.

Exemplary heteroaryl-ketone fused azadecalin compounds are described in U.S. Pat. No. 8,859,774; in U.S. Pat. No. 9,273,047; in U.S. Pat. No. 9,707,223; and in U.S. Pat. No. 9,956,216, all of which patents are hereby incorporated by reference in their entireties. In embodiments, the heteroaryl-ketone fused azadecalin GRA is the compound (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone (Example 18 of U.S. Pat. No. 8,859,774), also known as “relacorilant” and as “CORT125134”, which has the following structure:

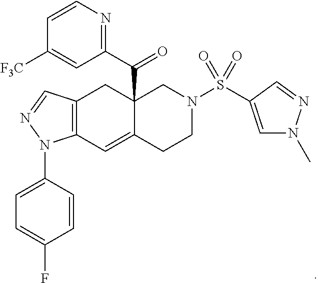 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

In embodiments, the heteroaryl-ketone fused azadecalin GRA is the compound (R)-(1-(4-fluorophenyl)-64(4-(trifluoromethyl)phenyl)sulfonyl)-4,4a,5,6,-7,8-hexahydro-1H-pyrazolo[3,4-g]isoquinolin-4a-yl)(thiazol-2-yl)methanone (termed “CORT122928”), which has the following structure:

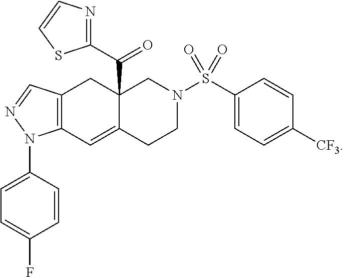 [SMILES: O=C(c1nccs1)[C@]12Cc3cnn(-c4ccc(F)cc4)c3C=C1CCN(S(=O)(=O)c1ccc(C(F)(F)F)cc1)C2]

In embodiments, the heteroaryl-ketone fused azadecalin GRA is the compound (R)-(1-(4-fluorophenyl)-6-((4-(trifluoromethyl)phenyl) sulfonyl)-4,4a, 5,6,7,8-hexahydro-1-H-pyrazolo P,4-g]isoquinolin-4a-yl) (pyridin-2-yl)methanone (also known as “dazucorilant” and as “CORT113176”), which has the following structure:

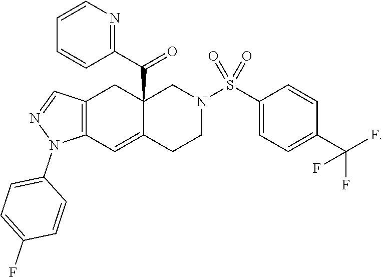 [SMILES: O=C(c1ccccn1)[C@]12Cc3cnn(-c4ccc(F)cc4)c3C=C1CCN(S(=O)(=O)c1ccc(C(F)(F)F)cc1)C2]

### Glucocorticoid Receptor Modulators (GRM)

Generally, treatment of Cushing's syndrome, Cushing's Disease, or a liver disorder can be provided by administering an effective amount of a glucocorticoid receptor modulator (GRM) of any chemical structure or mechanism of action. In embodiments, the GRM is mifepristone. In embodiments, the GRM is a selective GRM (SGRM). In embodiments, treatment of a liver disorder can be provided by administering an effective amount of a SGRM. In preferred embodiments, treatment of Cushing's syndrome, Cushing's Disease, or a liver disorder can be provided by administering an effective amount of a nonsteroidal SGRM. Provided herein are classes of exemplary GRMs, and in particular, exemplary nonsteroidal SGRMs, and specific members of such classes. However, one of skill in the art will readily recognize other related or unrelated GRMs and SGRMs that can be employed in the treatment methods described herein.

In some cases, the nonsteroidal SGRM is relacorilant (CORT125134), i.e., (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, which has the following structure:

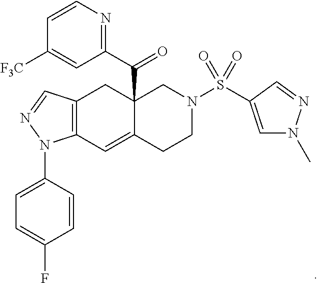 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Any suitable GRM dose may be used in the methods disclosed herein. The dose of GRM that is administered can be at least about 50 milligrams (mg) per day, or about 100 mg/day, e.g., about 150 mg/day, about 200 mg/day, about 250 mg/day, about 300 mg/day, about 350 mg/day, about 400 mg/day, about 450 mg/day, about 500 mg/day, or more. In embodiments, the GRM is administered orally. In some embodiments, the GRM is administered in at least one dose. In other words, the GRM can be administered in 1, 2, 3, 4, 5, 6, 7, 8, 9, 10 or more doses. In embodiments, the GRM is administered orally in 1, 2, 3, 4, 5, 6, 7, 8, 9, 10 or more doses.

The subject may be administered at least one dose of GRM in one or more doses over, for example, a 2-48 hour period. In some embodiments, the GRM is administered as a single dose. In other embodiments, the GRM is administered in more than one dose, e.g. 2 doses, 3 doses, 4 doses, 5 doses, or more doses over a 2-48 hour period, e.g., a 2 hour period, a 3 hour period, a 4 hour period, a 5 hour period, a 6 hour period, a 7 hour period, a 8 hour period, a 9 hour period, a 10 hour period, a 11 hour period, a 12 hour period, a 14 hour period, a 16 hour period, a 18 hour period, a 20 hour period, a 22 hour period, a 24 hour period, a 26 hour period, a 28 hour period, a 30 hour period, a 32 hour period, a 34 hour period, a 36 hour period, a 38 hour period, a 40 hour period, a 42 hour period, a 44 hour period, a 46 hour period or a 48 hour period. In some embodiments, the GRM is administered over 2-48 hours, 2-36 hours, 2-24 hours, 2-12 hours, 2-8 hours, 8-12 hours, 8-24 hours, 8-36 hours, 8-48 hours, 9-36 hours, 9-24 hours, 9-20 hours, 9-12 hours, 12-48 hours, 12-36 hours, 12-24 hours, 18-48 hours, 18-36 hours, 18-24 hours, 24-36 hours, 24-48 hours, 36-48 hours, or 42-48 hours.

Single or multiple administrations of formulations can be administered depending on the dosage and frequency as required and tolerated by the patient. The formulations should provide a sufficient quantity of active agent to effectively treat the disease state. Thus, in one embodiment, the pharmaceutical formulation for oral administration of a GRM is in a daily amount of between about 0.01 to about 150 mg per kilogram of body weight per day (mg/kg/day). In some embodiments, the daily amount is from about 1.0 to 100 mg/kg/day, 5 to 50 mg/kg/day, 10 to 30 mg/kg/day, and 10 to 20 mg/kg/day. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration.

The duration of treatment with a GRM or SGRM to treat Cushing's syndrome, Cushing's Disease, or a liver disorder can vary according to the severity of the condition in a subject and the subject's response to GRMs or SGRMs. In some embodiments, GRMs and SGRMs can be administered for a period of about 1 week to 104 weeks (2 years), more typically about 6 weeks to 80 weeks, most typically about 9 to 60 weeks. Suitable periods of administration also include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 104 weeks. Suitable periods of administration also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, 100, and 104 weeks. Generally administration of a GRM or SGRM should be continued until clinically significant reduction or amelioration is observed. Treatment with the GRM or SGRM in accordance with the methods disclosed herein may last for as long as two years or even longer.

In some embodiments, administration of a GRM or SGRM is not continuous and can be stopped for one or more periods of time, followed by one or more periods of time where administration resumes. Suitable periods where administration stops include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 100 weeks. Suitable periods where administration stops also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 weeks.

The dosage regimen also takes into consideration pharmacokinetics parameters well known in the art, i.e., the rate of absorption, bioavailability, metabolism, clearance, and the like (see, e.g., Hidalgo-Aragones (1996) J Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR modulator and disease or condition treated.

SGRMs can be used in combination with other active agents known to be useful in modulating a glucocorticoid receptor, or with adjunctive agents that may not be effective alone, but may contribute to the efficacy of the active agent.

In some embodiments, co-administration includes administering one active agent, a GRM or SGRM, within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a GRM as discussed herein has been formulated in an acceptable carrier, it can be placed in an appropriate container and labeled for treatment of an indicated condition. For administration of a GRM or SGRM, such labeling would include, e.g., instructions concerning the amount, frequency and method of administration.

The pharmaceutical compositions can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions for treating Cushing's syndrome, Cushing's Disease, or a liver disorder are useful for parenteral administration, such as intravenous (IV) administration or administration into a body cavity or lumen of an organ. The formulations for administration will commonly comprise a solution of the compositions dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the GRM compositions in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

### I. Combination Therapies

Various combinations with a GRM or SGRM and another agent (or a combination of such agents and compounds) may be employed to treat Cushing's syndrome, Cushing's Disease, or a liver disorder in the patient. By “combination therapy” or “in combination with”, it is not intended to imply that the therapeutic agents must be administered at the same time and/or formulated for delivery together, although these methods of delivery are within the scope described herein. The GRM or SGRM and the chemotherapeutic agent can be administered following the same or different dosing regimen. In some embodiments, the GRM or SGRM and the chemotherapeutic agent is administered sequentially in any order during the entire or portions of the treatment period. In some embodiments, the GRM or SGRM and the anticancer agent is administered simultaneously or approximately simultaneously (e.g., within about 1, 5, 10, 15, 20, or 30 minutes of each other). Non-limiting examples of combination therapies are as follows, with administration of the GRM or SGRM and the chemo agent for example, GRM or SGRM is “A” and the anticancer agent or compound, given as part of an chemo therapy regime, is “B”:

A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. Surgical intervention may also be applied in combination with the described therapy.

The present methods can be combined with other means of treatment such as surgery, radiation, targeted therapy, immunotherapy, or other treatments.

### EXAMPLES

The following examples are provided by way of illustration only and not by way of limitation. Those of skill will readily recognize a variety of noncritical parameters which could be changed or modified to yield essentially similar results.

### Example 1. Hepatic Impairment

A phase 1, open-label, multiple-dose study included 18 subjects (18-70 years). Of these subjects, 9 subjects had moderate hepatic impairment (Child-Pugh Class B) and 9 subjects were controls with normal hepatic function matched for age, sex, and body weight.

Relacorilant was administered at a dose of 300 mg/day for 10 days under fasted conditions. Blood samples for pharmacokinetic (PK) analysis were collected before dosing on Day 1 and from before dosing on Day 10 through 144 hours after the last dose of study drug (Day 16).

### Results: Hepatic Impairment Study

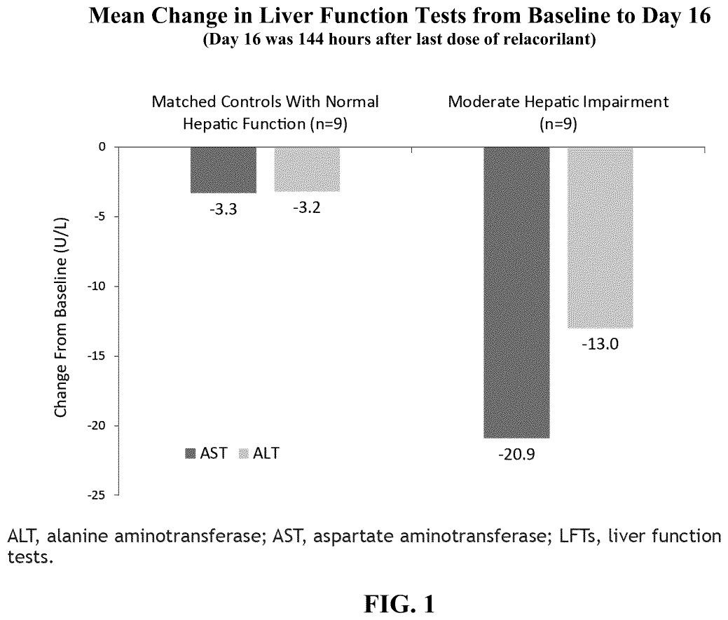

Subjects with moderate hepatic impairment were well matched for age, sex, and body weight Mean total Child-Pugh score of 7.9 (range: 7-9) in subjects with moderate hepatic impairment. Liver function was assessed by measurements of alanine aminotransferase (ALT) and aspartate aminotransferase (AST) levels. Reductions in mean liver function tests (LFTs) were observed in patients with moderate hepatic impairment. The mean change in LFTs from baseline to Day 16 (144 hours after last dose of relacorilant) are shown in FIG. 1.

Relacorilant Pharmacokinetic (PK): No apparent difference was observed between subjects with moderate hepatic impairment as compared to matched controls, despite relacorilant's primary hepatic route of elimination. The relacorilant exposures (as measured by area under the curve and maximum plasma concentration) largely overlapped across both groups.

### Example 2. Relacorilant-Itraconazole Drug-Drug Interaction Study

A phase 1, open-label, fixed-sequence crossover study (NCT03512548) included 25 healthy subjects (18-65 years). Subjects received relacorilant at a dose of 300 mg once per day for 10 days, followed by 10 days of receiving both relacorilant at a dose of 300 mg once per day with itraconazole at a dose of 200 mg once daily. Subjects with AST and/or ALT levels greater than 1.5 time the upper limit of normal (ULN) were excluded from the study.

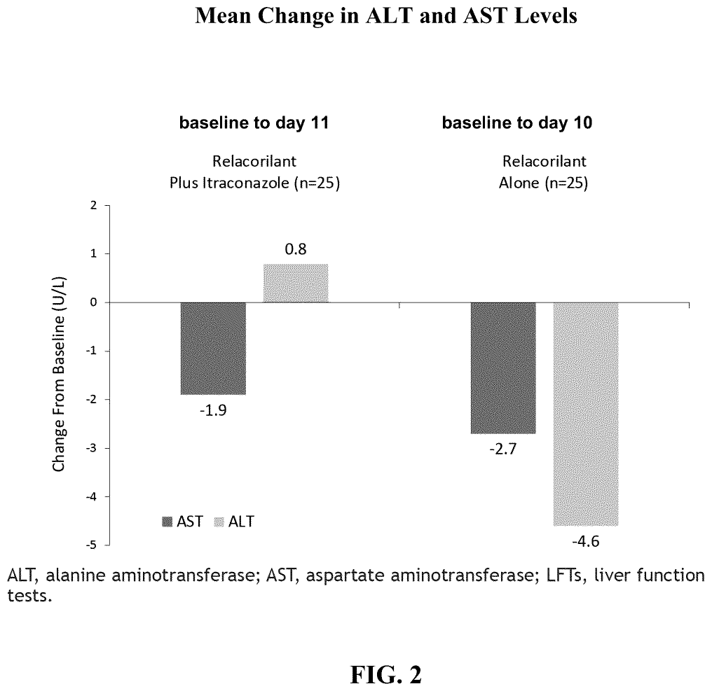

A trend toward AST reduction was observed among healthy adults treated with relacorilant plus intraconazole. (Itraconazole is an agent with reported liver toxicity.) Addition of itraconazole had no relevant effect on the adverse event profile of relacorilant in these subjects. Mean changes in LFTs in these subjects are shown in FIG. 2. The right-hand columns show the mean change in ALT and AST levels in subjects administered relacorilant alone (from baseline to Day 10); the left-hand columns show the mean changes in ALT and AST levels from baseline to Day 11 in subjects administered relacorilant plus itraconazole.

### Example 3. Phase 2 Cushing Syndrome Study

A multicenter, open-label study with two dose groups enrolled 34 patients (aged 18-80 years) with endogenous Cushing's Syndrome (CS) and impaired glucose tolerance or type 2 diabetes mellitus and/or uncontrolled or untreated hypertension. Patients with elevated AST or ALT (elevated defined as greater than 3-times the ULN) were excluded.

These patients were treated with relacorilant at two dosage levels. The dosages for each group were escalated in 50-mg dose increments every 4 weeks. The low dose group received relacorilant beginning at a dose of 100 mg/day, escalating to dosages of 200 mg/day during the duration of the 12-week treatment. The high-dose group received relacorilant beginning at a dose of 250 mg/day, escalating to a dosage of 400 mg/day during the duration of the 16-week treatment.

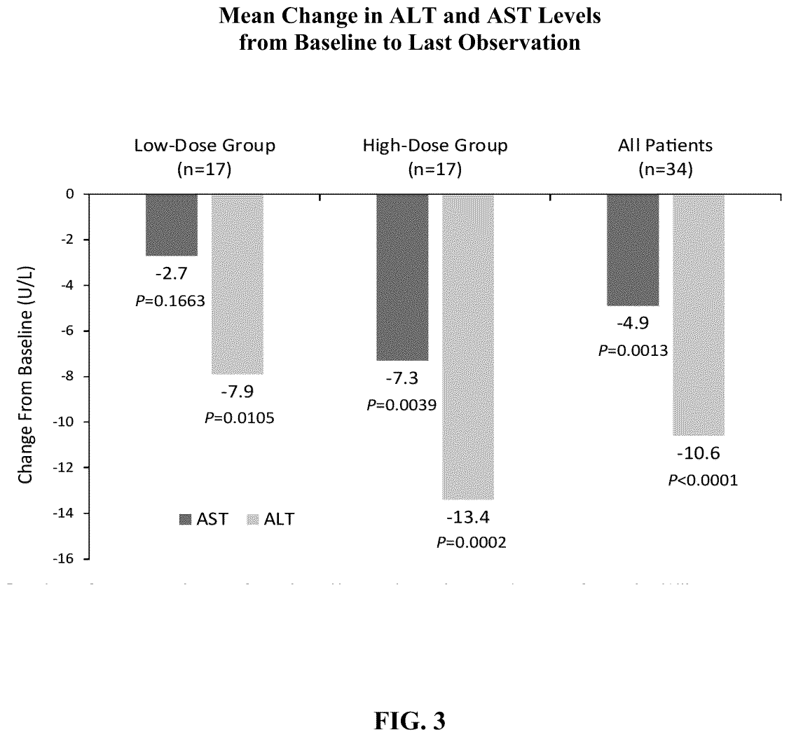

Reductions in LFTs were observed across both dose groups, with greater reductions in the high-dose group Normalization of ALT occurred in 2 of 4 patients with abnormal ALT values at baseline. These results are shown in FIG. 3, which presents the mean changes in LFTs from baseline to the last observation in the Efficacy Population (n=34). The left-most columns present the results from the patients in the Low-Dose group (n=17); the middle columns present the results from the patients in the High-Dose group (n=17); and the right-most columns present the combined results from all patients (n=34).

The P-values shown are the P-values for the mean change from baseline to last observation calculated from the Wilcoxon signed-rank test. The Efficacy Population includes all patients treated with relacorilant who had postbaseline data.

### Example 4. Ongoing Clinical Studies

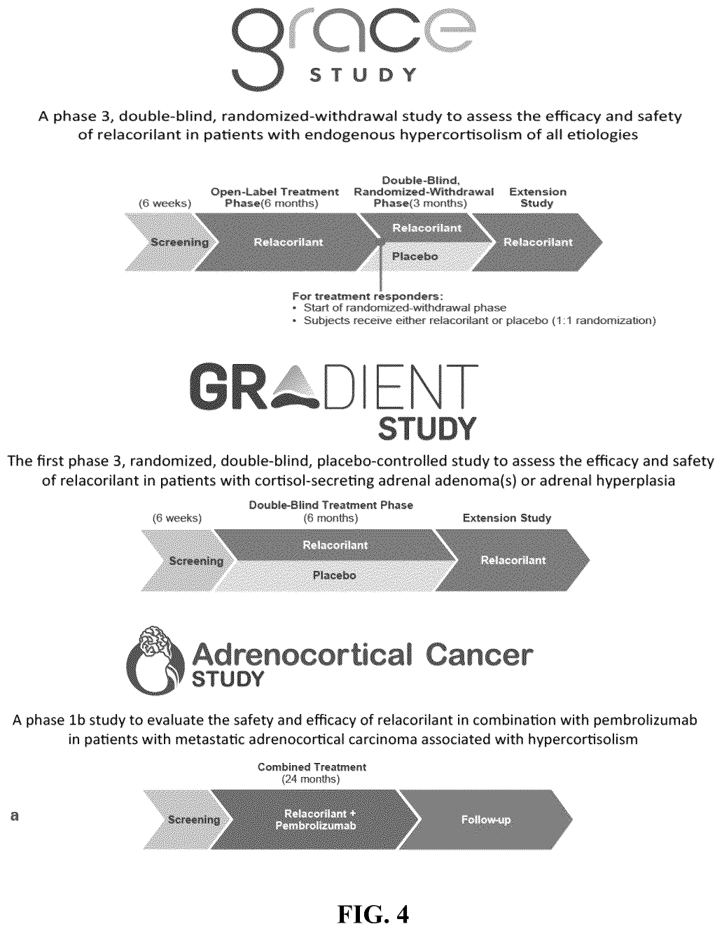

FIG. 4 presents graphical illustrations of the time-course and interventions for four, currently ongoing clinical studies of relacorilant in patients with hypercortisolism. The studies include two Phase 3 studies: GRACE (NCT03697109) and GRADIENT (NCT04308590), a Phase 2/3 long-term extension study (NCT03604198) in patients with CS of all etiologies, and a Phase 1b adrenocortical carcinoma study (NCT04373265). GRACE is a phase 3, double blind, randomized-withdrawal study of relacorilant in patients with endogenous hypercortisolism of all etiologies. GRADIENT is the first phase 3, randomized, double-blind, placebo-controlled study of relacorilant in patients with cortisol-secreting adrenal adenoma(s) or adrenal hyperplasia. The phase 2/3 extension study is a study of relacorilant in patients with endogenous hypercortisolism. The phase 1b study is a study to evaluate relacorilant in combination with pembrolizumab in patients with metastatic adrenocortical carcinoma associated with hypercortisolism.

All patents, patent publications, publications, and patent applications cited in this specification are hereby incorporated by reference herein in their entireties as if each individual publication or patent application were specifically and individually indicated to be incorporated by reference. In addition, although the foregoing has been described in some detail by way of illustration and example for purposes of clarity of understanding, it will be readily apparent to those of ordinary skill in the art in light of the teachings herein that certain changes and modifications may be made thereto without departing from the spirit or scope of the appended claims.

We claim:

1. A method of treating a patient suffering from a disorder selected from a liver disorder, Cushing's syndrome, Cushing's Disease, cancer, a fungal infection, a bacterial infection, a viral infection, an inflammatory disease or condition, a cardiovascular disease, an endocrine condition, and a kidney disease, and combinations thereof, without adverse effects on the liver of said patient,
the method comprising administering to the patient an effective amount of a nonsteroidal selective glucocorticoid receptor modulator (SGRM) compound comprising a heteroaryl ketone fused azadecalin structure to treat said disorder, or combination thereof, without adverse effects on the liver of the patient.

2. The method of claim 1, wherein the liver disorder is a fatty liver disease selected from alcohol related liver disease (ARLD) and nonalcoholic fatty liver disease (NAFLD).

3. The method of claim 2, wherein said alcohol-related liver disease (ARLD) is alcohol fatty liver disease (AFL), alcoholic steatohepatitis (ASH) or alcoholic cirrhosis.

4. The method of claim 2, wherein said nonalcoholic fatty liver disease (NAFLD) is nonalcoholic steatohepatitis (NASH) or nonalcoholic cirrhosis.

5. The method of claim 1, wherein the patient suffers from liver steatosis, wherein said administration of said nonsteroidal SGRM compound is effective to reduce liver steatosis in the patient.

6. The method of claim 1, wherein the patient suffers from Cushing's syndrome or Cushing's Disease.

7. The method of claim 6, wherein the patient suffers from a liver disorder.

8. The method of claim 1, wherein the nonsteroidal SGRM compound is relacorilant, (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, having the formula:

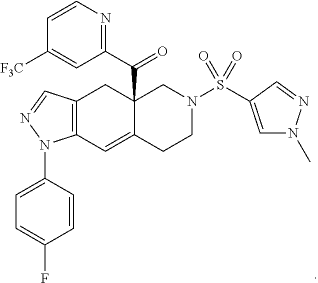 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

9. The method of claim 1, further comprising administering a further pharmaceutical composition to the patient, without further adverse effects on the liver of said patient.

10. The method of claim 9, wherein said further pharmaceutical composition comprises a CYP3A inhibitor selected from ketoconazole, itraconazole, nefazodone, ritonavir, nelfinavir, indinavir, boceprevir, clarithromycin, conivaptan, lopinavir, posaconazole, saquinavir, telaprevir, cobicistat, troleandomycin, tipranivir, paritaprevir and voriconazole.

11. The method of claim 9, wherein said further pharmaceutical composition comprises a drug selected from mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, exemestane, gefitinib, eltrombopag olamine, oxaliplatin, diltiazem, estramustine, peginterferon alfa-2b, methotrexate, cytarabine, maraviroc, mexiletine, and pentostatin.

12. A method of reducing or preventing liver toxicity in a patient receiving a drug which may cause liver toxicity, the method comprising administering to the patient an effective amount of a nonsteroidal selective glucocorticoid receptor modulator (SGRM) compound comprising a heteroaryl-ketone fused azadecalin structure in addition to said drug which may cause liver toxicity, without adverse effects on the liver of the patient.

13. The method of claim 12, wherein the patient suffers from a fatty liver disease selected from alcohol related liver disease (ARLD) and nonalcoholic fatty liver disease (NAFLD).

14. The method of claim 13, wherein said alcohol-related liver disease (ARLD) is alcohol fatty liver disease (AFL), alcoholic steatohepatitis (ASH) or alcoholic cirrhosis.

15. The method of claim 13, wherein said nonalcoholic fatty liver disease (NAFLD) is nonalcoholic steatohepatitis (NASH) or nonalcoholic cirrhosis.

16. The method of claim 12, wherein said patient suffers from an excess liver enzyme level in the blood of the liver enzyme alanine aminotransferase or of the liver enzyme aspartate aminotransferase, wherein said administration of said a nonsteroidal SGRM compound does not increase said liver enzyme levels in the blood.

17. The method of claim 12, wherein the patient suffers from Cushing's syndrome or Cushing's Disease.

18. The method of claim 12, wherein the nonsteroidal SGRM compound is relacorilant, (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, having the formula:

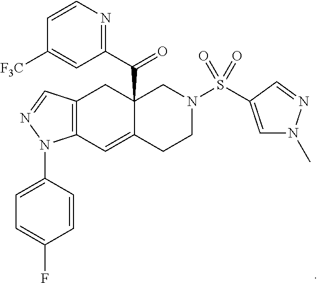 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

19. The method of claim 12, wherein the drug which may cause liver toxicity is a CYP3A inhibitor selected from ketoconazole, itraconazole, nefazodone, ritonavir, nelfinavir, indinavir, boceprevir, clarithromycin, conivaptan, lopinavir, posaconazole, saquinavir, telaprevir, cobicistat, troleandomycin, tipranivir, paritaprevir and voriconazole.

20. The method of claim 12, wherein the drug which may cause liver toxicity is a drug selected from mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, exemestane, gefitinib, eltrombopag olamine, oxaliplatin, diltiazem, estramustine, peginterferon alfa-2b, methotrexate, cytarabine, maraviroc, mexiletine, and pentostatin.

21. A method of treating a patient receiving a drug which may cause liver toxicity, without adverse effects on the liver of said patient, the method comprising administering to the patient an effective amount of a nonsteroidal selective glucocorticoid receptor modulator (SGRM) compound comprising a heteroaryl-ketone fused azadecalin structure to treat said patient receiving said drug which may cause liver toxicity, without adverse effects on the liver of the patient.

22. The method of claim 21, wherein the patient suffers from a fatty liver disease selected from alcohol related liver disease (ARLD) and nonalcoholic fatty liver disease (NAFLD).

23. The method of claim 22, wherein said alcohol-related liver disease (ARLD) is alcohol fatty liver disease (AFL), alcoholic steatohepatitis (ASH) or alcoholic cirrhosis.

24. The method of claim 22, wherein said nonalcoholic fatty liver disease (NAFLD) is nonalcoholic steatohepatitis (NASH) or nonalcoholic cirrhosis.

25. The method of claim 21, wherein the patient suffers from Cushing's syndrome or Cushing's Disease.

26. The method of claim 21, wherein the patient suffers from a liver disorder and from Cushing's syndrome or Cushing's Disease.

27. The method of claim 21, wherein the nonsteroidal SGRM compound is relacorilant, (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, having the formula:

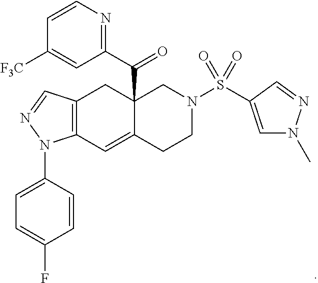 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

28. The method of claim 21, wherein the drug which may cause liver toxicity is a CYP3A inhibitor selected from ketoconazole, itraconazole, nefazodone, ritonavir, nelfinavir, indinavir, boceprevir, clarithromycin, conivaptan, lopinavir, posaconazole, saquinavir, telaprevir, cobicistat, troleandomycin, tipranivir, paritaprevir and voriconazole.

29. The method of claim 21, wherein the drug which may cause liver toxicity is a drug selected from mercaptopurine, indomethacin, phenytoin, rifampin, abacavir, allopurinol, amineptine, amiodarone, bicalutamide, chlorzoxazone, dactinomycin, dantrolene, diclofenac, diflunisal, fenoprofen, flutamide, hydroxyurea, imatinib, iproniazid, ketoconazole, labetalol, leflunomide, mefenamic acid, methyldopa, nefazodone, nitrofurantoin, perhexiline, propylthiouracil, stavudine, sulindac, tamoxifen, tizanidine, tolcapone, valproic acid, zidovudine, troglitazone, fluconazole, itraconazole, clomipramine, clarithromycin, testosterone, etodolac, pemoline, nevirapine, benzbromarone, busulfan, disulfiram, isoniazid, nimesulide, minocycline, alatrofloxacin mesylate, gemtuzumab ozogamicin, acetazolamide, benoxaprofen, bromfenac, danazol, febuxostat, griseofulvin, ibufenac, sunitinib, methimazole, sulfathiazole, terbinafine, ticrynafen, trovafloxacin, etravirine, tolvaptan, pazopanib, divalproex sodium, lumiracoxib, tasosartan, oxyphenisatin, tilbroquinol, alclofenac, aplaviroc, clomacran, dermatan, isaxonine, pipamazine, pralnacasan, sulfacarbamide, triacetyldiphenolisatin, fiduxosin, pafuraidine, phenoxypropazine, oxandrolone, acarbose, alpidem, bexarotene, voriconazole, bendazac, benzarone, benziodarone, atomoxetine, chlormezanone, erlotinib, cinchophen, tipranavir, clometacin, sorafenib, darunavir, cyclofenil, didanosine, interferon alfa-2b, interferon alfa-2a, recombinant, droxicam, ethambutol, infliximab, exifone, fialuridine, fipexide, fosphenytoin, gemcitabine, levofloxacin, mebanazine, moxisylyte, nialamide, nilutamide, niperotidine, nomifensine, nortriptyline, pirprofen, riluzole, ritonavir, sulcotidil, tolrestat, fenclozic acid, ebrotidine, nitrefazole, tetrabamate, xenazoic acid, zafirlukast, falnidamol, zimelidine, telithromycin, ximelagatran, duloxetine, glafenine, mepazine, lapatinib, alaproclate, orlistat, sitaxsentan, carbamazepine, felbamate, ciprofloxacin, oxymetholone, niacin, cyclosporine, albendazole, deferasirox, thiabendazole, raltegravir, dronedarone, bosentan, micafungin, bortezomib, milnacipran, asparaginase, efavirenz, interferon beta-1b, interferon beta-1a, interferon alfacon-1, lamotrigine, nandrolone decanoate, dacarbazine, acetaminophen, azathioprine, erythromycin, sulfasalazine, isotretinoin, atorvastatin, clozapine, 4-aminosalicylic acid, zileuton, acitretin, natalizumab, papaverine, gemfibrozil, ticlopidine, exemestane, gefitinib, eltrombopag olamine, oxaliplatin, diltiazem, estramustine, peginterferon alfa-2b, methotrexate, cytarabine, maraviroc, mexiletine, and pentostatin.

30. The method of claim 21, wherein said patient suffers from an excess liver enzyme level in the blood of the liver enzyme alanine aminotransferase or of the liver enzyme aspartate aminotransferase, wherein said administration of said a nonsteroidal SGRM compound does not increase said liver enzyme levels in the blood.
